(19) United States
(12) Patent Application Publication
(10) Pub. No.: US 2008/0056943 A1
(43) Pub. Date: Mar. 6, 2008

(54) Mechanism and method for collecting biomaterial samples and interfacing them to any device access port opening, or system, for the purposes of sample preparation, detection, manipulation, analysis or additional handling

(76) Inventor: John Wellington Sherwood, Seven Valleys, PA (US)

Correspondence Address: John W. Sherwood, 1060 Brighton Drive, Seven Valleys, PA, 17360, US

(21) Appl. No.: 11/897,094

(22) Filed: Aug. 30, 2007

Related U.S. Application Data
(60) Provisional application No. 60/841,635, filed on Sep. 1, 2006.

(51) Int. Cl. G01N 33/00

(52) U.S. Cl. 422/63

(57) ABSTRACT

Disclosed is an invention of a device mechanism and method containing an adhesive component for collecting DNA, RNA, enzymes, proteins, fungi, bacteria, viruses, or of any cellular or genetic material or any other bio or biochemical material (hereafter called biomaterial sample) and interfacing them to any accessible opening or access port(s) on any device or on any conceptually similar analysis system, for the purpose of sample preparation, detection, manipulation, and analysis of nucleotide polymorphisms, proteins, RNA, or of any further use of the biomaterial sample that can be collected by the sampling mechanism and method for the same purpose(s).

This patent filing claims the benefit of provisional application No. 60/841,635 filed on Sep. 1, 2006 in accordance with 35 U.S.C. §112 titled A mechanism and method for collecting biomaterial samples and presenting the samples to a device interface for further processing and/or for detection, manipulation, and analysis of nucleotides, proteins, RNA or of any cellular or genetic material.

### BACKGROUND OF THE INVENTION

1. Field of Invention

The present invention relates to a mechanism and method for collecting biomaterial samples (DNA, RNA, enzymes, proteins, fungi, bacteria, viruses, or of any cellular or genetic material or any other bio or biochemical material (hereafter called biomaterial sample) using an adhesive enabled surface and mechanically interfacing it to an opening or port on another device or system, for sample preparation, analysis, or any other form of handling.

2. Description of Background Art

Currently there is growing interest in analyzing biomaterials for all areas of biotechnology. Many industries such as the healthcare, agricultural, and security all need a means of rapidly collecting biomaterial samples and getting them into analysis systems and platforms. DNA and RNA sequencing technologies are also creating large amounts of data is later mined, by computer software. Many new applications are being developed that require a collection and processing infrastructure to provide important information to these analytical applications. That infrastructure is rapidly developing for traditional laboratory situations but there is a critical need for devices in remote point-of-care and point-of-use situations.

The need to determine information about biomaterials in the field, away from the laboratory or workbench, has been awaiting a reliable collection method/mechanism that can interface with analysis modules such as microfluidic chips, bioports and other interface embodiments. At the moment biosamples are typically collected by hand. They are placed in the appropriate shipping packaging and then sent to a laboratory for processing. Sample biomaterial is often collected by needles, creating a potential biohazard, and then later pipetted, by hand, in a laboratory for use in other areas for handling and analysis. Getting the samples into a handheld or remote system at the site of collection has not been adequately addressed.

The present invention enables a method to easily obtain a biosample in the field and to present it to several of a variety of interfaces for further processing. This eliminates errors in handling and collection as well as reduces the problem of sample contamination. This invention can adapt to accommodate a variety of biomaterial samples.

As used herein, the term “biomaterials” is defined as any biological sample collected by the adhesive bearing mechanism using this method of collection. The biomaterial can be a polymer of deoxyribonucleic acid (DNA), ribonucleic acid (RNA), enzymes, proteins, fungi, bacteria, viruses, any cellular or genetic material, any biofluid such as blood, saliva, or biochemical material.

The definition of “point-of-care” and “point-of-use”, used herein, refers to the actual collection environment such as a field emergency, hospital or doctors office, an agricultural field, or security check point, to name just a few examples.

“Microfluidics”, used herein, is defined as the bioengineering, biomechanical/microengineered machines scientific field that reduces macro-sized laboratory analysis and diagnostic functions onto substrates of specialized materials for the purpose of improving efficiency, reducing costs, and remote delivery of normally lab-sized functions into small, manageable, inexpensive, (and often) disposable devices.

As used herein, “bioports” is defined as a man-made reusable access opening to a living organism for diagnostic or therapeutic purposes. A bioport would, in theory, could be a very effective device in moving biomaterial into and out of numerous important physical environments.

The word “interface” used herein, is defined as the point at which two operations in an analytical or diagnostic system physically connect to each other. This physical connection functions as a method of exchanging biomaterial in preparation of a new operation such as sample preparation, cell lysis, amplification of the biomaterial, detection of attached fluorophore(s), as examples.

The present invention is a non-invasive, seemless, user friendly, mechanism and method for collection the biomaterial and presenting it to an interface of another component in the analytical and diagnostic device world, for additional operations or further use.

### BRIEF DESCRIPTION OF DRAWINGS

### DETAILED DESCRIPTION OF THE PREFERRED EMBODIMENTS

The features of the mechanism and method will be set forth in the description as follows, or may be learned from use of the invention.

It is to be understood that both the previous overview and description(s) and the following description(s) are examples of use and implementation. The scope of the inventions mechanism and method are defined by the claims.

One embodiment of the present invention involves non-invasively capturing DNA from skin cells. Using the direct pressure and downward movement of a disposable cartridge (comprised of a microfluidic chip containing the complete functionality for extracting DNA and analyzing single nucleotide polymorphisms) to activate the mechanism holding an adhesive containing surface against the surface of skin. On retraction of the collection mechanism, the adhesive collection surface is pulled closed against an access port on the microfluidic chip contained within the disposable cartridge. This seals the biomaterial over the extraction access port and prepares the environment necessary for sample preparation.

In the preferred embodiment, an adhesive prepared sampling surface 1 cm square adequately covers an access port area capable of capturing significant biomaterial for additional use in most microfluidic device applications.

Adhesive composition is tested in the cartridge design phase to determine potential reagent and biocompatibility levels. The preferred embodiment uses medical grade adhesive to reduce interference in downstream operations performed on the transferred biomaterial(s).

Methods and strategies for contamination control can be implemented within the microfluidic chip design level. This embodiment of the method for collecting biomaterial is quick and effective for a variety of applications. The biomaterial sample is collected, withdrawn into the protected environment of the cartridge, and sealed over the interface port of the next device. In this embodiment that device harbors an interface port, or conical well, on a microfluidic chip of approximately 2 cubic millimeters. Operations are then performed on the microfluidic chip, analysis output is generated, and the cartridge is then disposed.

The invention is comprised of several parts. A cylindrical rod has the sample collection surface (plate) on the end. This rod is housed in a spring-loaded channel. Side-rails, which connect both parts, push the sampling plate, containing the sampling media, down out of the sampling housing. In the current embodiment a molded latch structure opens the cartridge access cover as the sampling rod is being depressed. Immediately after collection the biomaterial sample the rod mechanism retracts and the cartridge access door closes. Attached to the access door on the interior side is the microfluidic chip. The chip then adheres to the mechanism's adhesive collection surface sealing the biomaterial over the access 2 mm port opening on the chip. Additional operations may then commence within the microfluidics chip.

The biomaterial is picked up by the sampling media and drawn, on the return of the rails, to it's original position. There it contacts the surface of a microfluidic chip, slide, or any port or device, or mechanism designed to process the cells or the genetic material of interest.

What is claimed is:

1. An adhesive coated, adhesive labeled or an adhesive tagged sampling mechanism for collecting and interfacing biomaterial samples to openings on biomaterial sample preparation, analysis, and handling devices or systems comprising;
a mechanically driven sampling mechanism component
a mechanically driven interface to a system preparing, analyzing, or performing handling of the collected biosample material.
an adhesive enabled surface or a device component

2. The collection method according to claim 1, wherein the biosample material is collected by a mechanically driven, adhesive coated, adhesive labeled, or adhesive tagged sampling surface.

3. The collection method according to claim 2, wherein the surface of a device component is adhesive enabled by any means such as two faced tape or direct application of adhesive to the collection component surface.

4. The collection method according to claim 2, wherein the adhesively enabled collection component is applied to a biosample by direct pressure.

5. The collection method according to claim 2, wherein adhesive is the primary collection mechanism for bringing biomaterial samples to openings on biomaterial sample preparation, analysis, and handling devices or systems.

6. The collection method according to claim 2, wherein a disposable cartridge uses an adhesive enabled sampling surface.

7. The interface according to claim 1, wherein the biosample material collected by the sampling mechanism covers and seals closed over the openings, or access port, of the sample preparation, sample analysis, sample handling device or system.

8. The interface according to claim 1, wherein a disposable cartridge includes the mechanical components to drive sample collection and directly interfaces with preparation, analysis or handling devices or systems.
